(19) United States
(12) Patent Application Publication
(10) Pub. No.: US 2025/0258174 A1
(43) Pub. Date: Aug. 14, 2025

(54) METHODS FOR PREDICTING THE RISK OF DEVELOPING MULTISYSTEM INFLAMMATORY SYNDROME (MIS) FOLLOWING EXPOSURE TO AN INFECTIOUS AGENT AND FOR DIAGNOSING MIS FOLLOWING EXPOSURE TO AN INFECTIOUS AGENT

(71) Applicants: SEMMELWEIS EGYETEM, Budapest (HU); HEIM PÁL ORSZÁGOS GYERMEKGYÓGYÁSZATI INTÉZET, Budapest (HU); HYCULT BIOTECH B.V., Uden (NL); TÁMOGATOTT KUTATÓCSOPORTOK IRODÁJA, Budapest (HU)

(72) Inventors: Erika KAJDÁCSI, Budapest (HU); György SINKOVITS, Budapest (HU); László CERVENAK, Budakeszi (HU); Zoltán PROHÁSZKA, Budapest (HU); Lisa HURLER, Wallerstein-Munzingen (DE); Veronika MARÁCZI, Budapest (HU); János Lajos SCHNUR, Budapest (HU); Erik Josephus Maria TOONEN, Nijmegen (NL); Jozephus Wilhelmus WILLEMS, Reek (NL)

(21) Appl. No.: 18/857,932

(22) PCT Filed: May 2, 2022
(86) PCT No.: PCT/HU2022/050039

§ 371 Date: Oct. 18, 2024

(30) Foreign Application Priority Data
Apr. 28, 2022 (HU) P2200135

(51) Int. Cl. G01N 33/573; G01N 33/68

(52) U.S. Cl. CPC G01N 33/573; G01N 33/6893; G01N 2333/8121; G01N 2333/96433; G01N 2800/52; G01N 2800/7095

(57) ABSTRACT

The invention relates to the diagnosis of multisystem inflammatory syndrome (MIS) following exposure to an infectious agent, to monitoring the efficacy of therapy against MIS, to a method for predicting the risk of developing MIS and to biological agents for use in the therapy of MIS associated with an exposure to an infectious agent.

The present application is the national stage of International Application PCT/HU2022/0050039, filed May 2, 2022.

This patent application has been supported by the MSCA-ITN (Horizon 2020) “CORVOS” Grant (Grant number 860044).

### FIELD OF THE INVENTION

The invention relates to the diagnosis of multisystem inflammatory syndrome (MIS) following exposure to an infectious agent, to monitoring the efficacy of therapy against MIS, to a method for predicting the risk of developing MIS and to biological agents for use in the therapy of MIS associated with an exposure to an infectious agent.

### BACKGROUND OF THE INVENTION

Multisystem inflammatory syndrome (MIS) can affect children and adolescents (MIS-C) and adults (MIS-A). MIS is a rare but serious condition associated with infections, often with viral infections, in which different body parts become inflamed, including the heart, lungs, kidneys, brain, skin, eyes, or gastrointestinal organs. Multisystem inflammatory syndrome in children and adolescents (MIS-C) is a rare (5.1/1 million person-months in the general population; and 316/1 million person-months in SARS-CoV-2 infected), severe, potentially life-threatening complication of SARS CoV2 infection Payne et al. Incidence of Multisystem Inflammatory Syndrome in Children Among US Persons Infected With SARS-CoV-2. JAMA Netw Open. 2021 Jun. 1; 4 (6):e2116420.9) MIS-C typically occurs in 8-12-year-old children several weeks after exposure to, or infection with SARS CoV2, even if the disease was asymptomatic (Abrams et al. Factors linked to severe outcomes in multisystem inflammatory syndrome in children (MIS-C) in the USA: a retrospective surveillance study. Lancet Child Adolesc Health. 2021 May; 5(5):323-331). Presenting clinical features include fever, rash, mucositis, conjunctivitis, cardiac complications, and hypotension or shock. The disease develops with high fever, marked inflammation and shock-like picture several weeks after exposure to, or mild infection with SARS-CoV-2. Deep immune profiling identified activated macrophages, neutrophils, B-plasmablasts and CD8+T cells as key determinants of pathogenesis together with multiple inflammatory markers. Almost all of the affected cases are anti-spike IgG seropositive indicating reconvalescent stage, but most of them are viral RNA negative in nasal swabs making viral persistence less plausible as trigger of the condition. The optimal treatment of MIS-C is currently unknown, intravenous immunoglobulin (IVIG) preparations (with or without corticosteroids) are widely used to good effect rapidly leading to resolving symptoms.

Complement has been implicated to play a role in the pathogenesis of MIS-C. Diorio and colleagues measured the terminal pathway activation marker sC5b-9 in 6 children with MIS-C for the first time and observed a trend for higher values (Multisystem inflammatory syndrome in children and COVID-19 are distinct presentations of SARS-CoV-2. J Clin Invest. 2020 November 2; 130 (11): 5967-5975). Elevated levels of sC5b-9 were also reported in 18 MIS-C cases, comparable to concentrations observed in severe pediatric COVID-19 (Diorio et al. Evidence of thrombotic microangiopathy in children with SARS-CoV-2 across the spectrum of clinical presentations. 2020 December 8; 4 (23): 6051-6063). When analyzed together, no association between anti-SARS CoV receptor-binding domain (RBD) antibody levels and sC5b-9 was observed suggesting that anti-SARS CoV2 immune complexes are likely not the activating factors behind complement activation in these patients. However, activation of the classical complement pathway has formally not been investigated in this study. In their serum proteomic profiling study Porritt et al. observed upregulated heavy and light chains of immunoglobulins and C1QA, ClQB and ClQC proteins in severe MIS-C cases, without further investigation of complement activation in that cohort (The autoimmune signature of hyperinflammatory multisystem inflammatory syndrome in children. J Clin Invest. 2021 October 15; 131 (20): e151520). The study of Syrimi and coworkers (The immune landscape of SARS-CoV-2-associated Multisystem Inflammatory Syndrome in Children (MIS-C) from acute disease to recovery. iScience Volume 24, Issue 11, 19 Nov. 2021, 103215) investigated the sC5b-9 together with other complement protein and regulator levels in 16 cases with MIS-C. Authors observed that C9 and Factor I levels were increased, and the terminal complement pathway was activated in MIS-C, but analysis of potential triggers behind this activation (like anti-SARS-CoV2-antibodies) was not performed. The role of circulating immune complexes behind complement activation was formally investigated by the group of Hoste et al. (Hoste L et al. TIM3+ TRBV11-2 T cells and IFNγ signature in patrolling monocytes and CD16+ NK cells delineate MIS-C. J Exp Med. 2022 Feb. 7; 219 (2):e20211381. doi: 10.1084/jem.20211381.), who analyzed diluted plasma samples of 10 MIS-C cases and 6 healthy controls. Although a clear trend for increased levels of complement proteins and activation markers was present, statistically significant differences were not observed (except for the elevation of C1-inhibitor). Multiple complement activation markers were determined by Hoste et al. and partly showed to be elevated, but they failed to observe significant differences between acute MIS-C and healthy controls The above pioneer studies providing the first implications about the potential involvement of complement in the pathogenesis of MIS-C used multi-omic technologies on a few samples to describe the immune landscape of this disease. Detailed analysis of the presence and potential triggers of complement activation, involving all of the pathways, was not performed until now. Lastly, the effect of treatment on the complement system was not analyzed in the context of disease severity and anti-SARS-CoV-2 antibody response.

### SUMMARY OF THE INVENTION

### In a First Aspect:

A method is provided for diagnosing multisystem inflammatory syndrome (MIS) associated with an exposure to an infectious agent in a subject or for identifying a person as being at an increased risk of developing MIS associated with an exposure to an infectious agent, comprising
measuring the level of at least one biomarker selected from the group consisting of: C1s-C1-INH complex, neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, Troponin T, pro-BNP, complement classical pathway activity, complement alternative pathway activity, and properdin in a body fluid sample of the subject or person, wherein an altered level of the at least one biomarker indicates that the subject has MIS or that the person is at an increased risk of developing MIS, respectively.

The method is preferably for diagnosing MIS. In another preferred embodiment the method is for identifying a person as being at an increased risk of developing MIS.

A method is provided for determining whether a patient has a positive response to therapy for MIS associated with an exposure to an infectious agent, comprising
(A) measuring the levels of at least one biomarker selected from the group consisting of: C1s-C1-INH complex, neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, Troponin T, pro-BNP, complement classical pathway activity, complement alternative pathway activity, and properdin at a time point (I) and a time point (II) in body fluid samples of the patient,
(B) comparing the levels obtained in (A) to a control level which is the same for time point (I) and time point (II), wherein a lesser deviation from a control level of the biomarker at the time point (II) than at the time point (I) indicates that the patient has a positive response to the therapy,
wherein (I) is earlier in therapy than (II).

Preferably, the therapy is IVIG therapy.

A method is provided for treating a patient suffering in MIS associated with an exposure to an infectious agent who is insufficiently responding to IVIG therapy,
comprising
(i) identifying the patient as insufficiently responding to IVIG therapy, comprising
(A) measuring levels of at least one biomarker selected from the group consisting of: C1s-C1-INH complex, neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, Troponin T, pro-BNP, complement classical pathway activity, complement alternative pathway activity, and properdin at a time point (I) and a time point (II) in body fluid samples of the patient, wherein (I) is earlier in IVIG therapy than (II),
and
(B) comparing the levels obtained in (A) to a control level which is the same for time point (I) and time point (II), wherein a greater or equivalent deviation from the control level of the biomarker at time point (II) compared to the deviation from the control level of the biomarker at time point (I) indicates that the patient is non-responsive to IVIG therapy
and
(ii) administering a therapeutical agent selected from the group consisting of glucocorticoids and biological agents to the patient.

A biological agent for use is provided in the treatment of a patient having MIS associated with an exposure to an infectious agent and insufficiently responding to IVIG therapy, wherein said use comprises the administration of the biological agent to the patient and wherein the patient is identified as insufficiently responding to IVIG therapy, comprising
(A) measuring levels of at least one biomarker selected from the group consisting of: C1s-C1-INH complex, neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin at a time point (I) and a time point (II) in body fluid samples of the patient, wherein (I) is earlier in IVIG therapy than (II), and
(B) comparing the levels obtained in (A) to a control level which is the same for time point (I) and time point (II),
wherein a greater or equivalent deviation from the control level of the biomarker at time point (II) compared to the deviation from the control level of the biomarker at time point (I) indicates that the patient is non-responsive to the therapy.

An altered level of the biomarker may be an elevated or decreased level compared to a reference or control level.

The reference or control level is preferably a level calculated from values of the biomarker characteristic of healthy individuals. The reference or control level is preferably the level of the biomarker characteristic of healthy individuals. The reference or control level or the values from which the reference or control level is calculated is/are preferably measured by the same method as the altered level. An altered level preferably means that the level is significantly different from the reference or control level, that is e.g. there is an at least 10% or at least 15% or at least 20% or at least 25% or at least 30% deviation from the reference or control level or preferably the altered level is statistically significantly different from the reference or control level.

In a highly preferred embodiment a C1s-C1-INH complex level measured in a subject that is at least 120% or at least 125% or at least 130% or at least 135% or most preferably at least about 140% of the reference level indicates that the subject has MIS.

In a highly preferred embodiment a Bb level measured in a subject that is at least 120%, at least 130%, at least 140%, at least 150%, at least 160% or most preferably at least about 175% or about 177% of the reference level indicates that the subject has MIS.

In a highly preferred embodiment a neopterin level measured in a subject that is at least 500%, at least 750%, at least 1000%, at least 1250% or most preferably at least about 1400% or 1450% of the reference level indicates that the subject has MIS.

In a highly preferred embodiment a measured C3a level that is at least 150% or at least 175% or at least 200% or most preferably at least 240% of the reference level indicates that the subject has MIS.

In a highly preferred embodiment a measured sC5b-9 level that is at least 150%, at least 175%, at least 200% or most preferably at least about 220% of the reference level indicates that the subject has MIS.

Preferably the measured level of the biomarker is compared to a reference or control level of the biomarker characteristic of healthy individuals, that is, individuals not having MIS or not at an increased risk of developing MIS, or individuals who were not exposed to the infectious agent within 4 weeks, preferably within 6 weeks, preferably within 8 weeks or—where appropriate—individuals who are already in remission from MIS. The reference or control level is preferably the reference or control level characteristic of the age of the subject to be tested.

The MIS might be MIS in children and adolescents (MIS-C) or in adults (MIS-A), preferably MIS-C.

The infectious agent is preferably a virus, more preferably Severe Acute Respiratory Syndrome-Coronavirus 2 (SARS-CoV-2).

Preferably the body fluid sample is blood sample, more preferably plasma sample, most preferably EDTA-plasma.

Preferably,
an elevated level of the at least one biomarker selected from the group consisting of: C1s-C1-INH complex, neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, Troponin T, pro-BNP,
   and/or an decreased level of properdin,
   and/or an altered level of the at least one biomarker selected from the group consisting of complement classical pathway activity and complement alternative pathway activity
   indicates that the subject has MIS or that the person is at an increased risk of developing MIS.

Preferably,
a lower level of the at least one biomarker selected from the group consisting of: C1s-C1-INH complex, neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP
and/or a higher level of properdin,
at a time point (II) than at time point (I) in a body fluid sample of the patient,
and/or a lesser deviation from a control level of the at least one biomarker selected from the group consisting of complement classical pathway activity and complement alternative pathway activity at time point (II) than at time point (I)
indicates that the patient has a positive response to the therapy.

Preferably,
a higher or equivalent level of the at least one biomarker selected from the group consisting of: C1s-C1-INH complex, neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, Troponin T, pro-BNP, at time point (II) than at time point (I);
and/or a lower or equivalent level of properdin at time point (II) than at time point (I);
and/or a greater or equivalent deviation from the control level of the at least one biomarker selected from the group consisting of complement classical pathway activity and complement alternative pathway activity at time point (II) compared to the deviation from the control level of the biomarker at time point (I),
indicates that the patient is non-responsive to the therapy.

In a preferred embodiment the at least one biomarker is selected from a group consisting of: C1s-C1-INH complex, neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, Troponin T, pro-BNP and properdin.

In a preferred embodiment the at least one biomarker is selected from a group consisting of: C1s-C1-INH complex, neopterin, Bb, C3a, sC5b-9.

In a preferred embodiment the at least one biomarker is selected from a group consisting of: C1s-C1-INH complex, neopterin, Bb.

In a preferred embodiment the at least one biomarker is C1s-C1-INH complex, neopterin and Bb.

In a preferred embodiment the at least one biomarker is: C1s-C1-INH complex and Bb. In a preferred embodiment the at least one biomarker is: C1s-C1-INH complex and neopterin. In a preferred embodiment the at least one biomarker is Bb and neopterin. In a preferred embodiment the at least one biomarker is C1s-C1-INH complex. In a preferred embodiment the at least one biomarker is Bb. In a more preferred embodiment the at least one biomarker is neopterin.

Preferably, the biological agent is an anti-cytokine activity agent, preferably an interleukin [IL]-1 antagonist, an IL-6 receptor blocker or an anti-tumour necrosis factor agent. Preferably the anti-cytokine activity agent is selected from infliximab, anakinra and tocilizumab. Preferably the anti-cytokine activity agent is infliximab. Preferably the anti-cytokine activity agent is tocilizumab. Most preferably the anti-cytokine activity agent is anakinra.

Preferably the subject or patient was exposed to SARS-CoV-2 more than 1 week or more than 10 days or preferably more than 2 weeks, more preferably more than 3 weeks, preferably more than 4 weeks before the method is performed.

Preferably the subject or patient was exposed to SARS-CoV-2 less than 6 months, less than 5 months, less than 4 months and more preferably less than 3 months, preferably no more than 12, 11, 10, 9, 8, 7, 6, 5, 4, 3, 2, 1 weeks before the method is performed.

In a preferred embodiment, a method is provided for diagnosing MIS-C associated with an exposure to SARS-CoV-2 in a subject, comprising
measuring the level of a biomarker selected from C1s-C1-INH complex, neopterin, Bb, preferably neopterin,
in a plasma sample derived from the subject,
   wherein an elevated level of the biomarker indicates that the subject has MIS-C,
wherein the subject has been exposed to SARS-CoV-2 prior to measuring the level of the biomarker in their plasma sample.

In another preferred embodiment, a method is provided for identifying a person as being at an increased risk of developing MIS-C associated with an exposure to SARS-CoV-2, comprising
measuring the level of a biomarker selected from C1s-C1-INH complex, neopterin, Bb, preferably neopterin,
in a plasma sample derived from the subject,
   wherein an elevated level of the biomarker indicates that the person is at an increased risk of developing MIS-C,
wherein the person has been exposed to SARS-CoV-2 prior to measuring the level of the biomarker in their plasma sample.

In another preferred embodiment a method is provided for determining whether a patient has a positive response to therapy for MIS-C associated with an exposure to SARS-CoV-2, comprising
(A) measuring levels of at least one biomarker selected from C1s-C1-INH complex, neopterin, Bb, preferably neopterin,
at a time point (I) and a time point (II) in body fluid samples of the patient,
(B) comparing the levels obtained in (A) to a control level which is the same for time point (I) and time point (II),
   wherein
   (I) is earlier in therapy than (II).

The term “insufficient response” or “insufficiently responding” refers to cases where the condition of the patient does not improve (e.g. the severity/diversity/frequency of the symptoms does not decrease) or does not improve as expected or does not significantly improve in spite of receiving the therapy. The term “insufficient response” or “insufficiently responding” also refers to cases where the condition of the patient requires a change in therapy (e.g. where the condition of the patient improves too slowly or some of the symptoms remain too severe to be treated with the therapy any longer). The terms “insufficient response” or “insufficiently responding” also refer to “no response” or “non-responding”, respectively.

### BRIEF DESCRIPTION OF THE DRAWINGS

FIG. 1. Demographic and clinical data of the MIS-C cohort. A: Study samples included shown by clinical stage. The left axis shows number of cases, case numbers (n) written on the right sum up cases falling into the various sample combination groups. B: 7-days moving average of daily new COVID-19 cases in Hungary (orange columns, left y axis), as identified by PCR or rapid antigen testing (source: koronavirus.gov.hu, downloaded 27-01-2022). Darker line: Kernel density estimation (KDE) plot fitted on the MIS-C cases admitted to the Heim Pal National Pediatric Institute and University of Szeged between 25-11-2020 and 27-08-2021. Lighter grey line: KDE plot fitted on COVID-19 exposures for 24/34 MIS-C cases, for whom information on contact was available. The KDE curves indicate the estimated number of COVID-19 exposures or MIS-C cases, respectively, expressed as cases per 30 days (right axis). C: Age (solid symbols: males, open symbols: females) and disease severity indicators of MIS-C cases (for hospital stay solid diamonds: days hospitalized, open diamonds days in PICU). For calculation of vasoactive inotrope score see the materials and methods section.

FIG. 2. Disease severity marker levels before and after IVIG therapy in the MIS-C cohort. A: Pre- and >10 days post-IVIG treatment (denoted acute and remission) absolute blood counts and clinical laboratory results of the 34 MIS-C cases. The horizontal lines indicate the age-specific reference ranges. Since no established reference range was available for neopterin, results of healthy controls are shown for this marker, together with individual changes of levels during IVIG treatment and in remission. P value symbols (** p<0.01, *** p<0.001) indicate significant results after 5% false discovery rate correction using the Benjamini-Hochberg method. B: Principal component analysis loading plot of the top 7 features contributing to principal components one and two. Loading of length of fever, PICU treatment and extent of multisystem involvement (indicated by boxes) are shown as supplementary variables. C: Heatmap of correlation matrix of clinical and laboratory markers of MIS-C. Clinical score indicates to total number of skin- (rash, feet and hands signs), mucosal- (conjunctivitis, cheilits, oral mucositis), gastrointestinal- (diarrhea, vomiting, abdominal pain) and cardiac involvement. Color intensity-coding indicates the strength of each correlation (Spearman correlation coefficients) with asterisks indicating significance (* indicate p<0.003 significant results, the limit was obtained after 5% false discovery rate correction using the Benjamini-Hochberg method). Non-significant differences are not marked.

FIG. 3. Detailed complement profile of the MIS-C cohort before, during and after IVIG therapy, and of healthy controls. Levels of the complement pathway (classical-, lectin- and alternative) activities, factors (C1q, C4, C3, Factors D, B and properdin) and regulators (C1-inhibitor antigen and activity, Factors H and I) were determined in serum, whereas that of activation products (C1s-C1-inhibitor complex, MASP1-C1-inhibitor complex, C4a, C4d, C3a, Bb and sC5b-9) in EDTA plasma. Violin plots (with dashed horizontal lines indicating the median values, dotted lines indicating the quartiles) show results for the 18 healthy control children (HC), and for all MIS-C cases who had samples from acute stage, before IVIG treatment, or from stable remission after hospital discharge. Grey lines show changes of marker levels for individual MIS-C cases over the course of disease: before (‘acute’) and right after IVIG therapy (‘IVIG’), and in remission (‘remission’; some patients had two samples taken in remission, for case numbers in the individual groups see the flow chart in FIG. 1). P values for the pair-wise group comparisons (HC-acute and acute-remission groups) on the violin plots were calculated by the Mann-Whitney test, whereas for the line charts p values were obtained by the Wilcoxon matched-pairs ranked sum test. Non-significant differences are not marked, and p value symbols (* p<0.05, ** p<0.01, *** p<0.001) indicate significant results after 5% false discovery rate correction using the Benjamini-Hochberg method.

FIG. 4. Associations between complement markers, clinical, laboratory and anti-SARS-CoV-2 humoral immune response features in the MIS-C cohort. Heatmap of correlation matrix of complement markers (Panel A), complement markers and clinical and laboratory features (Panel B), complement markers and SARS-CoV-2 humoral immune response measures (Panel C). Color intensity-coding indicates the strength of each correlation (Spearman correlation coefficients) with asterisks indicating significance (* indicate on Panel A p<0.007, on Panel C p<0.005 significant results, the limit was obtained after 5% false discovery rate correction using the Benjamini-Hochberg method).

FIG. 5. Association between biochemical markers of complement and anti-SARS-CoV2 antibody response, macrophage activation, acute phase reaction and clinical severity of MIS-C. A: Violin plots (with horizontal lines indicating the median values) show results for the MIS-C cases that had samples from acute stage, before IVIG treatment, or from stable remission after hospital discharge. Grey lines show changes of marker levels for individual MIS-C cases from before treatment (‘acute’) after hospital discharge in remission (‘remission”). P values for the line charts were obtained by the Wilcoxon matched-pairs ranked sum test. Non-significant differences are not marked, and p value symbols (* p<0.05, ** p<0.01, * ** p<0.001) indicate significant results after 5% false discovery rate correction using the Benjamini-Hochberg method. B: Violin plots showing classical- (C1s-C1-inhibitor complex), alternative- (Bb fragment) and terminal pathway (sC5b-9 complex) activation marker levels in groups with low (below median) or high (above median) anti-SARS-CoV-2 spike and nucleocapsid IgG levels (Generic assay IgG binding index), macrophage activation (ferritin levels) or acute phase reaction (CRP concentration). * indicates p=0.002, considered significant after 5% false discovery rate correction using the Benjamini-Hochberg method.

FIG. 6. Analysis of complement- and macrophage activation markers for diagnosis verification and therapy monitoring purposes in MIS-C
A: Cumulative frequencies (%) of MIS-C cases in acute (red, solid circles) or in remission (purple, open circles) stage, and of healthy controls (blue, open rhombus), plotted against the range of selected complement- and macrophage activation markers. B: Diagnostic performance of complement activation marker and neopterin levels to differentiate acute MIS-C versus MIS-C in remission. C: Area under the curve (AUC) values with 95% confidence intervals.

FIGS. 7A and B. Diagnostic performance of complement activation marker and neopterin levels to differentiate healthy controls versus acute MIS-C (left panels) and acute MIS-C versus remission MIS-C (right panels).

FIG. 8. Receiver operating characteristic curves for predicting acute MIS-C are shown. Area under the curve (AUC) values with 95% confidence intervals are presented in Tables 3 and 4.

### DETAILED DESCRIPTION OF THE INVENTION

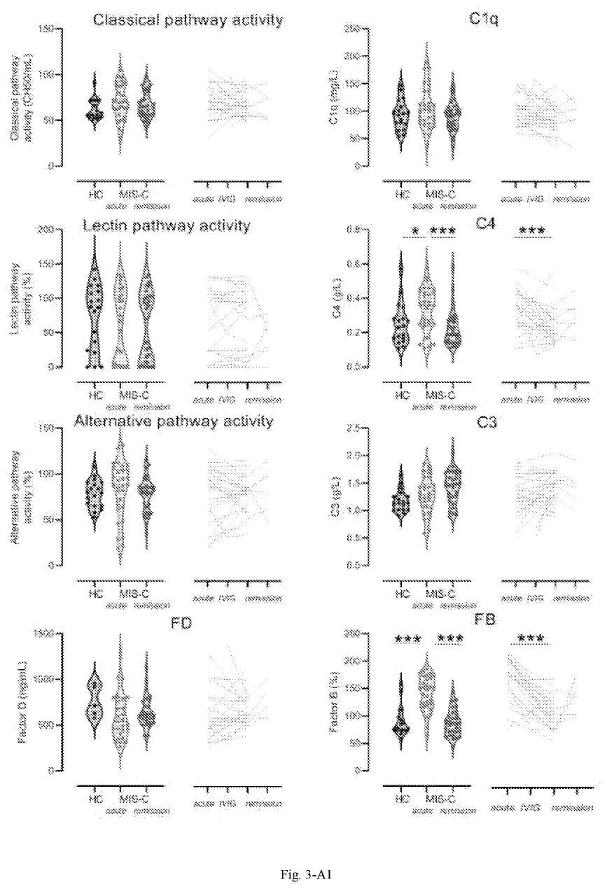

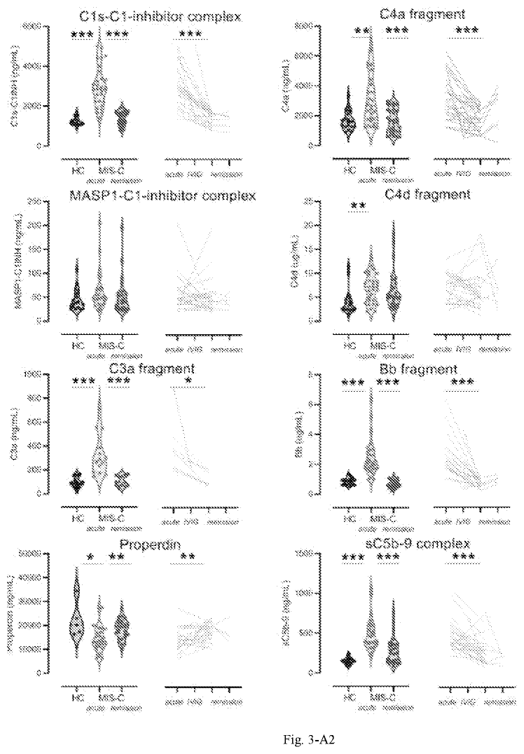

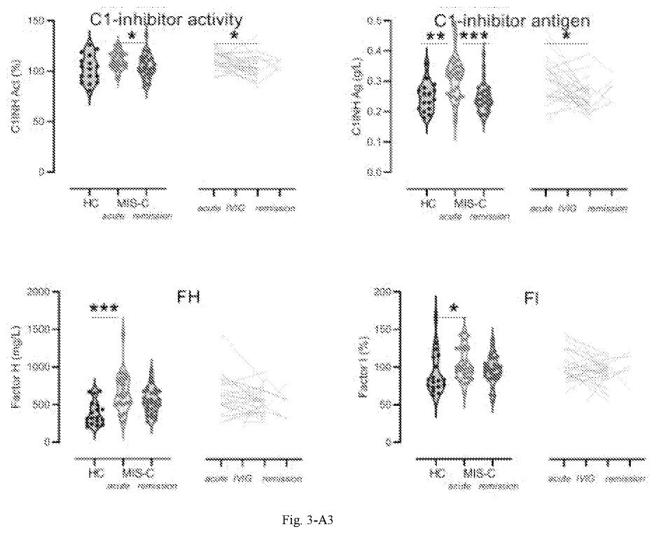

Several complement activation products were identified as appropriate markers to verify the clinical diagnosis of MIS-C (not only terminal pathway marker sC5b-9 as suggested by previous studies), and to show therapy efficacy after IVIG treatment. Acute MIS-C was found to be characterized by elevation of C1s-C1-inhibitor complex, C4a and C4d, activation markers of the C1 complex and joint classical and lectin pathways, by increase in Bb and C3a concentrations, indicating ongoing activation and amplification of the central component, C3, of the system, and finally, by elevation of sC5b-9, the activation product of the cell-damaging terminal pathway. All of these activation product levels (except C4d) showed rapid and sharp decline after treatment by IVIG, together with additional markers of inflammation (FIG. 3). These results indicate that activation of complement is part of the systemic inflammatory response in MIS-C with rapid resolution after the immunomodulatory therapy. Macrophage activation (ferritin) was shown to be associated with the group of Bb, C3a and sC5b-9 activation products suggesting an interaction between these factors in the development of MIS-C. Surprisingly, there was no relationship between anti-SARS CoV2 humoral immune response features and activation of the classical pathway, indicating that other factors than immunoglobulins might give rise to the elevation of C1s-C1-inhibitor complex. Rather unexpectedly, it was also revealed that neopterin, a small molecule marker belonging to the group of pteridines, has 100% sensitivity and 100% specificity to differentiate between healthy controls and MIS-C, and association with resolution of inflammation has the same diagnostic performance.

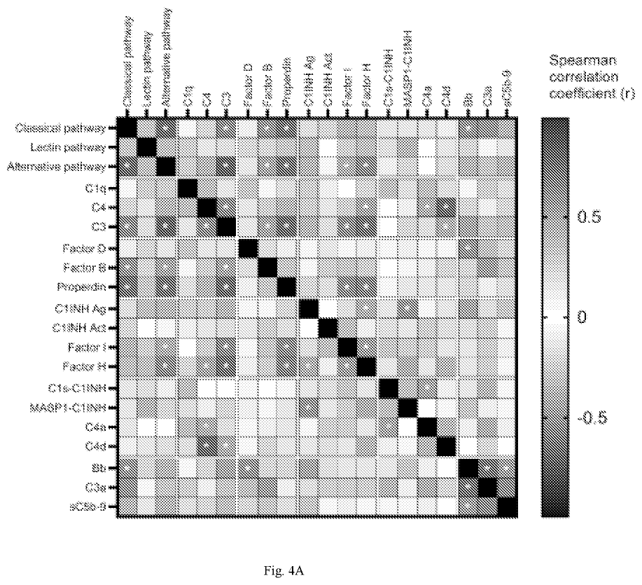

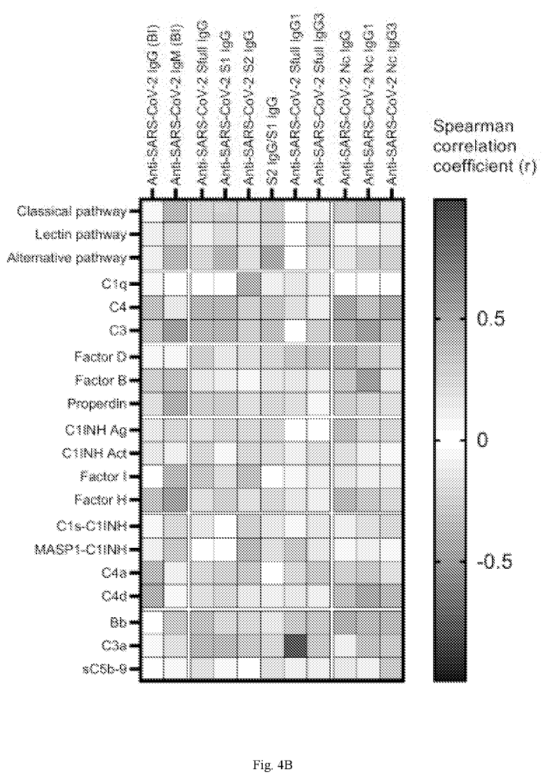

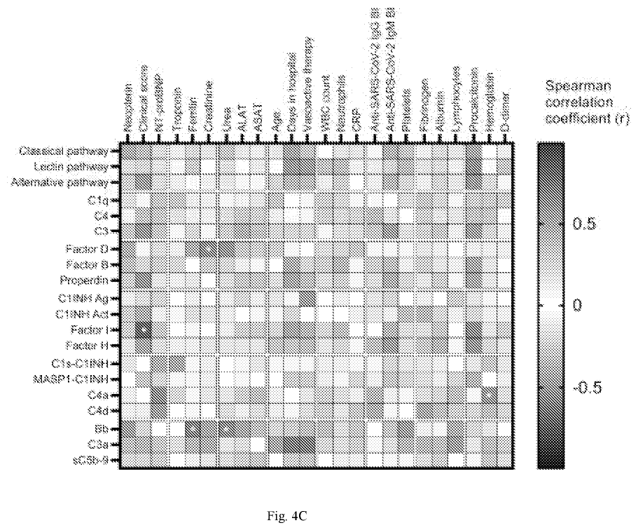

The prototypic macrophage activation marker ferritin and AP marker Bb were both elevated and showed significant association in acute MIS-C (FIGS. 4C and 6), with rapid and uniform decrease after immunomodulatory therapy. The results may indicate that alternative pathway- and macrophage activation are parallel and/or related processes in MIS-C.

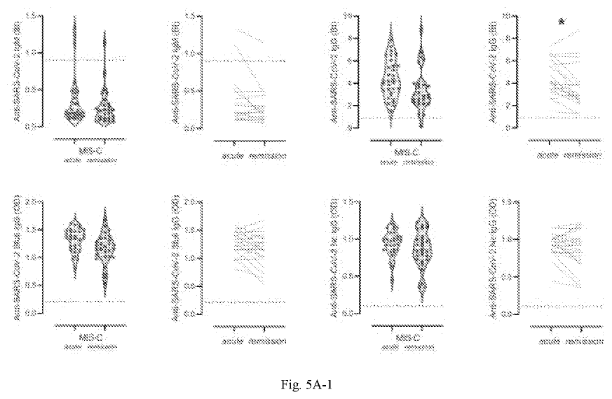

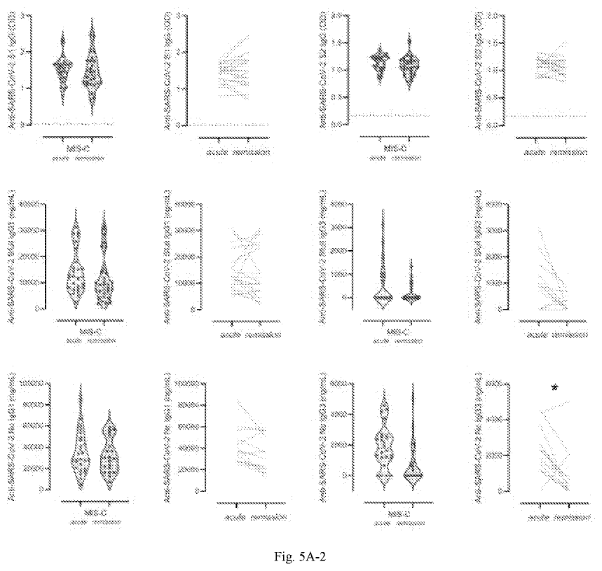

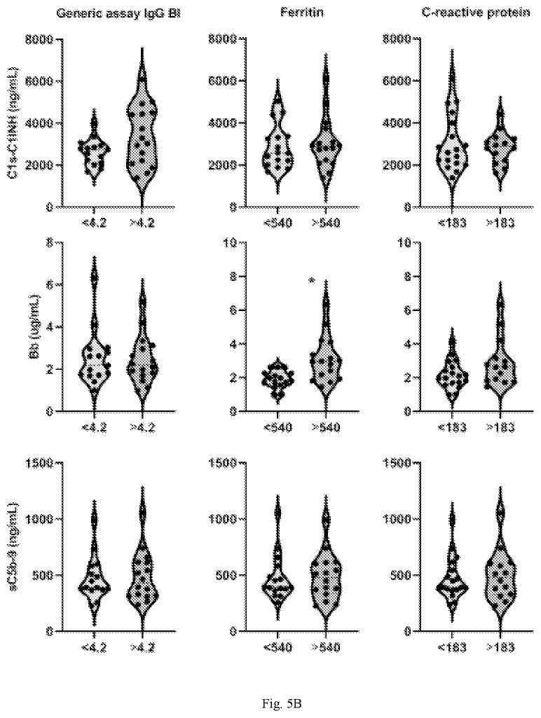

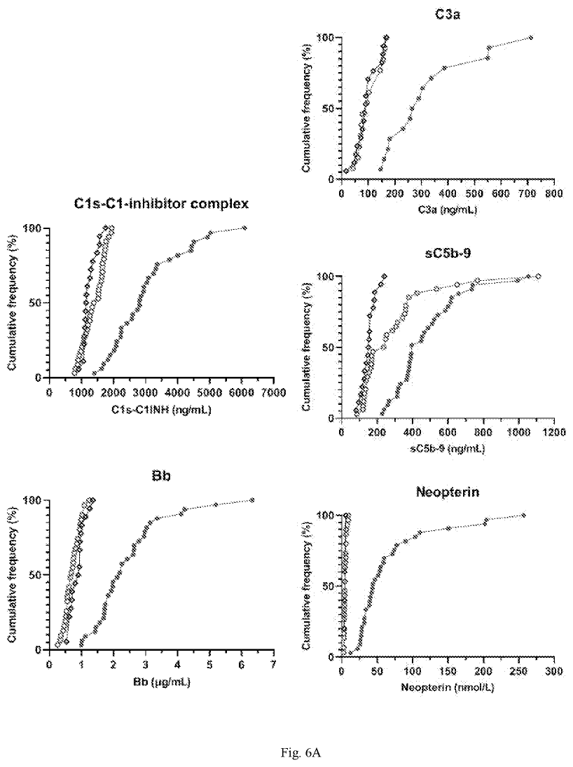

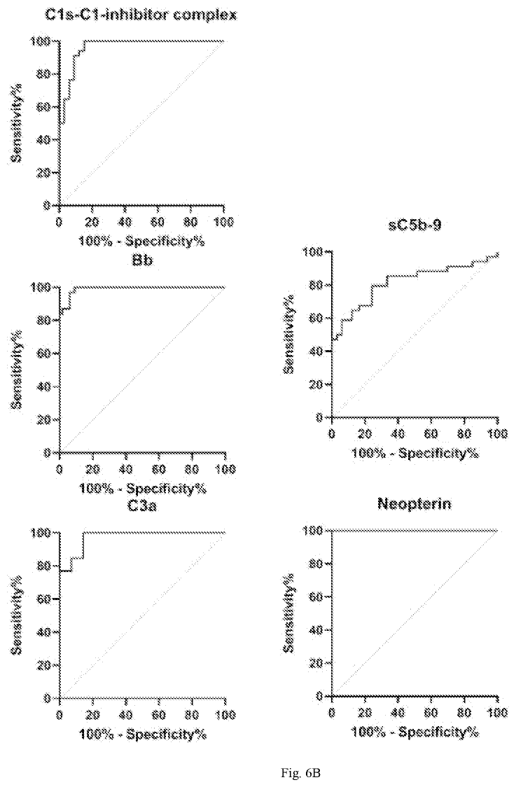

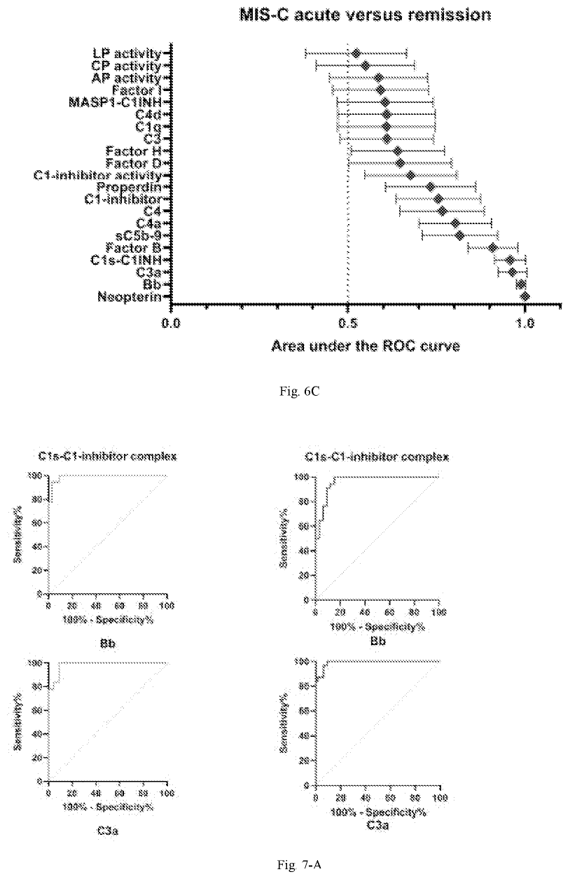

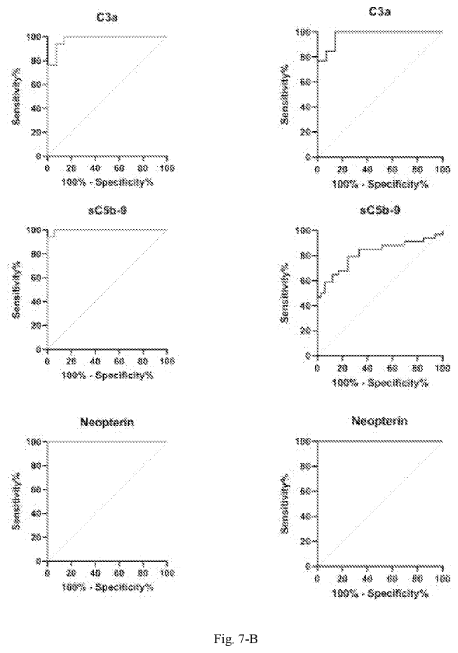

The lack of correlation between any of the complement activation markers, including all markers of the classical pathway, and features of the anti-SARS-CoV-2 humoral immune response is surprising. Together with the lack of clinically meaningful decline of anti-SARS-CoV-2 antibodies after immunomodulatory therapy (acute and remission stage, grey line charts on FIG. 5A) these observations make it unlikely that anti-SARS-CoV-2 antibodies play a major role in the initiation and sustainment of multisystemic inflammation in these patients.

Activation of both C4 and Factor B, but not that of the early activation marker of lectin pathway (MASP1-C1-inhibitor complex) was demonstrated, supporting the conclusion on the activation of both classical and alternative pathways (elevated C4a, C4d, Bb, FIG. 3) in MIS-C.

Ferritin and neopterin levels were highly significantly elevated in acute disease stage, and showed uniform sharp decline with resolution of the hyperinflammation syndrome.

Levels of neopterin, a member of the chemical group named as pteridines, has never been investigated in MIS-C before. It is noteworthy that neopterin is synthesized by macrophages in response to interferon gamma (Werner E R et al. Interferon-gamma-induced degradation of tryptophan by human cells in vitro. Biol Chem Hoppe Seyler. 1987 October; 368(10):1407-12. doi: 10.1515/bchm3.1987.368.2.1407. PMID: 3122784.), and showed highest and most uniform difference in acute stage of MIS-C, when compared to both healthy controls or remission stage. Among children with MIS-C immunomodulatory therapy with application of intravenous immunoglobulins and corticosteroids show high efficacy (McArdle A J et al. Treatment of Multisystem Inflammatory Syndrome in Children. N Engl J Med. 2021 Jul. 1; 385(1):11-22. doi: 10.1056/NEJMoa2102968. Epub 2021 June 16. PMID: 34133854; PMCID: PMC8220965.) and only a few of the patients required additional interleukine-1 inhibitory treatment (Lee P Y et al. Distinct clinical and immunological features of SARS-CoV-2-induced multisystem inflammatory syndrome in children. J Clin Invest. 2020 Nov. 2; 130(11):5942-5950. doi: 10.1172/JC1141113. PMID: 32701511; PMCID: PMC7598077.). IVIG is a pooled preparation of normal IgG obtained from several thousand healthy donors. The mechanisms of action of IVIG are complex, including several non-exclusive mechanisms affecting soluble molecules and cellular constituents of the immune system. Molecular targets of IVIG therapy depend on the Fc or F(ab′)2 parts of immunoglobulins, making cell-surface expressed receptors (mainly Fc-receptors) and soluble mediators (including complement) as key players.

The term biological agent refers to a substance that is made from a living organism or its products and is used in the prevention, diagnosis, or treatment of a disease. Biologic agents include for example antibodies, interleukins, oligonucleotides, proteins, anti-cytokine activity agents.

Complement component names are given is Table 1.

TABLE 1
Official names, abbreviations and alternative names of
complement components, regulators, activation products

| | | | |
|---|---|---|---|
| Official name* | Abbreviation | Alternative names | Comments |

Pathways

| | | | |
|---|---|---|---|
| Complement classical | CP | Classical pathway | |
| pathway | | | |
| Complement lectin | LP | Lectin pathway | MBL-lectin pathway |
| pathway | | | |
| Complement alternative | AP | Alternative pathway | |
| pathway | | | |
| Complement terminal | TP | Terminal pathway | C5, C6, C7, C8 and C9 |
| pathway | | | |

Protein complexes

| | | | |
|---|---|---|---|
| Complement component | C1 | C1 complex, C1qr₂s₂, | |
| 1 | | C1qC1r₂C1s₂ complex | |
| soluble complmenet | sC5b-9 | terminal complement | Complement terminal |
| terminal pathway | | complex (TCC), | pathway activation |
| complex | | membrane attack | product |
| | | complex (MAC), soluble | |
| | | MAC (sMAC), sC5b-9 | |
| | | complex | |
| C1s-C1-inhibitor | C1s-C1-INH complex | C1s/C1-INH complex | early classical pathway |
| complex | | | activation marker |
| MASP-1-C1-inhibitor | MASP-1-C1-INH | MASP-1/C1-INH | early lectin pathway |
| complex | complex | complex | activation marker |

Proteins

| | | | |
|---|---|---|---|
| Complement component | C1q | Complement C1q | Component of the C1 |
| 1q | | | complex |
| Complement component | C1r | Complement C1r | Component of the C1 |
| 1r | | | complex |
| Complement component | C1s | Complement C1s | Component of the C1 |
| 1s | | | complex |
| Complement component | C3 | Complement C3 | |
| 3 | | | |
| Complement fragment | C3a | Anaphylatoxin C3a, | Split product of C3 |
| C3a | | Complement C3a | |
| Complement component | C4 | Complement C4 | |
| 4 | | | |
| Complement fragment | C4a | Complement component | Split product of C4 |
| C4a | | C4a, Complement C4a | |
| Complement fragment | C4d | Complement component | Split product of C4 |
| C4d | | C4d, Complement C4d | |
| Complement component | C9 | Complement C9 | |
| 9 | | | |
| Factor B | FB | Complement factor B | |
| Factor D | FD | Complement factor D | |
| Factor H | FH | Complement factor H | |
| Factor I | FI | Complement Factor I | |
| C1 esterase inhibitor | C1-INH | C1-inhibitor, C1 | |
| | | inhibitor, C1Inh | |
| Complement fragment | Bb | Factor Bb, Complement | Alternative pathway |
| Bb | | Factor Bb | activation product, split |
| | | | product of Factor B |
| Mannose-binding lectin | MBL | Mannan-binding lectin, | |
| | | Mannan-binding protein | |
| | | (MBP) | |
| Properdin | Properdin | | no abbreviation |
| MBL-associated serine | MASP-1 | | |
| protease 1 | | | |

*Based on the official IUIS complement nomenclature

In a second aspect, a method is provided for diagnosing multisystem inflammatory syndrome (MIS) associated with an exposure to an infectious agent in a subject or for identifying a person as being at an increased risk of developing MIS associated with an exposure to an infectious agent, comprising
(I) measuring the level of C1s-C1-INH complex
   in a body fluid sample of the subject,
and
(II) measuring the level of at least one biomarker selected from a group (A) consisting of: neopterin, Bb, C3a, sC5b-9, C1-INH antigen, C1-INH activity, C4a, C4d, Factor B, Factor D, Factor H, Factor I, C-reactive protein, haptoglobin, Troponin, ferritin, D-dimer, pro-BNP, Troponin T, pro-BNP, complement classical pathway activity, complement alternative pathway activity, and properdin,
   in a body fluid sample of the subject,
wherein an altered level of C1s-C1-INH complex and an altered level of the at least one biomarker indicates that the subject has MIS or that the person is at an increased risk of developing MIS, respectively.

Preferably,
an elevated level of C1s-C1-INH complex
and
   an elevated level of the at least one biomarker selected from the group consisting of neopterin, Bb, C3a, sC5b-9,
   C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP,
and/or a decreased level of properdin,
and/or an altered level of the at least one biomarker selected from the group consisting of complement classical pathway activity, complement alternative pathway activity
indicates that the subject has MIS or that the person is at an increased risk of developing MIS.

The method is preferably for diagnosing MIS. In another preferred embodiment the method is for identifying a person as being at an increased risk of developing MIS.

A method is provided for determining whether a patient has a positive response to therapy for MIS associated with an exposure to an infectious agent, comprising
(α) measuring the levels of C1s-C1-INH complex at a time point (I) and a time point (II) in body fluid samples of the patient, and
(β) measuring levels of at least one biomarker selected from a group (A) consisting of: neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin at a time point (I) and a time point (II) in body fluid samples of the patient,
wherein
a lesser deviation from a control level of C1s-C1-INH complex and the at least one biomarker selected from group (A) at time point (II) than at time point (I) indicates that the patient has a positive response to the therapy, wherein
(I) is earlier in therapy than (II).

Preferably,
a lower level of C1s-C1-INH complex at a time point (II) than at time point (I) in body fluid samples of the patient, and a lower level of the at least one biomarker selected from the group consisting of: neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP at a time point (II) than at time point (I) in body fluid samples of the patient,
and/or a higher level of properdin, at a time point (II) than at time point (I) in body fluid samples of the patient, and/or a lesser deviation from a control level of the at least one biomarker selected from the group consisting of complement classical pathway activity, complement alternative pathway activity at time point (II) than at time point (I) indicates that the patient has a positive response to the therapy.

Preferably, the therapy is IVIG therapy.

A method is provided for treating a patient suffering in MIS associated with an exposure to an infectious agent who is insufficiently responding to IVIG therapy,
comprising
(i) identifying the patient as insufficiently responding to IVIG therapy, comprising
(α) measuring the levels of C1s-C1-INH complex at a time point (I) and a time point (II) in body fluid samples of the patient, and
(β) measuring levels of at least one biomarker selected from a group (A) consisting of: neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity and properdin at a time point (I) and a time point (II) in body fluid samples of the patient, wherein (I) is earlier in IVIG therapy than (II),
wherein a greater or equivalent deviation from the control level of C1s-C1-INH complex and the at least one biomarker selected from group (A) at time point (II) compared to the deviation from the control level of the biomarker at time point (I) indicates that the patient is non-responsive to IVIG therapy and
   (ii) administering a therapeutical agent selected from the group consisting of glucocorticoids and biological agents to the patient.

A biological agent for use is provided in the treatment of a patient having MIS associated with an exposure to an infectious agent and insufficiently responding to IVIG therapy, wherein said use comprises the administration of the biological agent to the patient and wherein the patient is identified as insufficiently responding to IVIG therapy, comprising
(α) measuring the levels of C1s-C1-INH complex at a time point (I) and a time point (II) in body fluid samples of the patient, and
(β) measuring levels of at least one biomarker selected from a group (A) consisting of: neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin at a time point (I) and a time point (II) in body fluid samples of the patient,
wherein (I) is earlier in IVIG therapy than (II),
wherein a greater or equivalent deviation from the control level of C1s-C1-INH complex the at least one biomarker selected from group (A) at time point (II) compared to the deviation from the control level of the biomarker at time point (I) indicates that the patient is non-responsive to IVIG therapy.

Preferably,
a higher or equivalent level of C1s-C1-INH complex
and a higher or equivalent level of the at least one biomarker selected from the group consisting of: neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, at time point (II) than at time point (II);
and/or a lower or equivalent level of properdin at time point (II) than at time point (II);
and/or a greater or equivalent deviation from the control level of the at least one biomarker selected from the group consisting of complement classical pathway activity, complement alternative pathway activity at time point (II) compared to the deviation from the control level of the biomarker at time point (I), indicates that the patient is non-responsive to IVIG therapy.

In a preferred embodiment group (A) consists of: neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP and properdin.

In a preferred embodiment group (A) consists of: neopterin, Bb, C3a, sC5b-9.

In a third aspect, a method is provided for diagnosing multisystem inflammatory syndrome (MIS) associated with an exposure to an infectious agent in a subject or for identifying a person as being at an increased risk of developing MIS associated with an exposure to an infectious agent, comprising
(I) measuring the level of Bb in a body fluid sample of the subject, and
(II) measuring the level of at least one biomarker selected from a group (B) consisting of: C1s-C1-INH complex, neopterin, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin in a body fluid sample of the subject, wherein an altered level of Bb and an altered level of the at least one biomarker indicates that the subject has MIS or that the person is at an increased risk of developing MIS, respectively.

Preferably,
an elevated level of Bb
and
   an elevated level of the at least one biomarker selected from the group consisting of C1s-C1-INH complex, neopterin, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP,
and/or a decreased level of properdin,
and/or an altered level of the at least one biomarker selected from the group consisting of complement classical pathway activity, complement alternative pathway activity indicates that the subject has MIS or that the person is at an increased risk of developing MIS.

The method is preferably for diagnosing MIS. In another preferred embodiment the method is for identifying a person as being at an increased risk of developing MIS.

A method is provided for determining whether a patient has a positive response to therapy for MIS associated with an exposure to an infectious agent, comprising
(α) measuring the levels of Bb at a time point (I) and a time point (II) in body fluid samples of the patient, and
(β) measuring levels of at least one biomarker selected from a group (B) consisting of: C1s-C1-INH complex, neopterin, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin
   at a time point (I) and a time point (II) in body fluid samples of the patient,
wherein
a lesser deviation from a control level of Bb and the at least one biomarker selected from group (B) at time point (II) than at time point (I) indicates that the patient has a positive response to the therapy,
wherein (I) is earlier in therapy than (II).

Preferably,
a lower level of Bb at a time point (II) than at time point (I) in body fluid samples of the patient, and a lower level of the at least one biomarker selected from the group consisting of: neopterin, C1s-C1-INH complex, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP at a time point (I) than at time point (II) in body fluid samples of the patient,
and/or a higher level of properdin, at a time point (II) than at time point (I) in body fluid samples of the patient, and/or a lesser deviation from a control level of the at least one biomarker selected from the group consisting of complement classical pathway activity, complement alternative pathway activity at time point (II) than at time point (I) indicates that the patient has a positive response to the therapy.

Preferably, the therapy is IVIG therapy.

A method is provided for treating a patient suffering in MIS associated with an exposure to an infectious agent who is insufficiently responding to IVIG therapy,
comprising
(i) identifying the patient as insufficiently responding to IVIG therapy, comprising
(α) measuring the levels of Bb at a time point (I) and a time point (II) in body fluid samples of the patient, and
(β) measuring levels of at least one biomarker selected from a group (B) consisting of: C1s-C1-INH complex, neopterin, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin at a time point (I) and a time point (II) in body fluid samples of the patient,
wherein (I) is earlier in IVIG therapy than (II),
wherein a greater or equivalent deviation from the control level of Bb and the at least one biomarker selected from group (B) at time point (II) compared to the deviation from the control level of the biomarker at time point (I) indicates that the patient is non-responsive to IVIG therapy and
   (ii) administering a therapeutical agent selected from the group consisting of glucocorticoids and biological agents to the patient.

A biological agent for use is provided in the treatment of a patient having MIS associated with an exposure to an infectious agent and insufficiently responding to IVIG therapy, wherein said use comprises the administration of the biological agent to the patient and wherein the patient is identified as insufficiently responding to IVIG therapy, comprising
(α) measuring the levels of Bb at a time point (I) and a time point (II) in body fluid samples of the patient, and
(β) measuring levels of at least one biomarker selected from a group (B) consisting of: C1s-C1-INH complex, neopterin, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin at a time point (I) and a time point (II) in body fluid samples of the patient,
wherein (I) is earlier in IVIG therapy than (II),
wherein a greater or equivalent deviation from the control level of C1s-C1-INH complex and the at least one biomarker selected from group (B) at time point (II) compared to the deviation from the control level of the biomarker at time point (I) indicates that the patient is non-responsive to IVIG therapy.

Preferably,
a higher or equivalent level of Bb at time point (II) than at time point (I);
and a higher or equivalent level of the at least one biomarker selected from the group consisting of: neopterin, C1s-C1-INH complex, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, at time point (II) than at time point (II);
and/or a lower or equivalent level of properdin at time point (II) than at time point (I);
and/or a greater or equivalent deviation from the control level of the at least one biomarker selected from the group consisting of complement classical pathway activity, complement alternative pathway activity at time point (II) compared to the deviation from the control level of the biomarker at time point (I), indicates that the patient is non-responsive to IVIG therapy.

In a preferred embodiment group (B) consists of: neopterin, C1s-C1-INH complex, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP and properdin.

In a preferred embodiment group (B) consists of: neopterin, C1s-C1-INH complex, C3a, sC5b-9.

In a fourth aspect, a method is provided for diagnosing multisystem inflammatory syndrome (MIS) associated with an exposure to an infectious agent in a subject or for identifying a person as being at an increased risk of developing MIS associated with an exposure to an infectious agent, comprising
(I) measuring the level of neopterin in a body fluid sample of the subject, and
(II) measuring the level of at least one biomarker selected from a group (C) consisting of: C1s-C1-INH complex, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin in abody fluid sample of the subject,
wherein an altered level of neopterin and an altered level of the at least one biomarker selected from group (C) indicates that the subject has MIS or that the person is at an increased risk of developing MIS, respectively.

Preferably,
an elevated level of neopterin
and
   an elevated level of the at least one biomarker selected from the group consisting of C1s-C1-INH complex, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP,
and/or a decreased level of properdin,
and/or an altered level of the at least one biomarker selected from the group consisting of complement classical pathway activity, complement alternative pathway activity indicates that the subject has MIS or that the person is at an increased risk of developing MIS.

The method is preferably for diagnosing MIS. In another preferred embodiment the method is for identifying a person as being at an increased risk of developing MIS.

A method is provided for determining whether a patient has a positive response to therapy for MIS associated with an exposure to an infectious agent, comprising
(α) measuring the levels of neopterin at a time point (I) and a time point (II) in body fluid samples of the patient, and
(β) measuring levels of at least one biomarker selected from a group (C) consisting of: C1s-C1-INH complex, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin at a time point (I) and a time point (II) in body fluid samples of the patient,
wherein
a lesser deviation from a control level of neopterin and the at least one biomarker selected from group (C) at time point (II) than at time point (I) indicates that the patient has a positive response to the therapy,
wherein
(I) is earlier in therapy than (II).

Preferably,
a lower level of neopterin at a time point (II) than at time point (I) in body fluid samples of the patient,
and a lower level of the at least one biomarker selected from the group consisting of: Bb, C1s-C1-INH complex, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, and/or a higher level of properdin at a time point (II) than at time point (I) in body fluid samples of the patient,
and/or a lesser deviation from a control level of the at least one biomarker selected from the group consisting of complement classical pathway activity, complement alternative pathway activity at time point (II) than at time point (I) indicates that the patient has a positive response to the therapy.

Preferably, the therapy is IVIG therapy.

A method is provided for treating a patient suffering in MIS associated with an exposure to an infectious agent who is insufficiently responding to IVIG therapy, comprising
(i) identifying the patient as insufficiently responding to IVIG therapy, comprising
(α) measuring the levels of neopterin at a time point (I) and a time point (II) in body fluid samples of the patient, and
(β) measuring levels of at least one biomarker selected from a group (C) consisting of: C1s-C1-INH complex, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin at a time point (I) and a time point (II) in body fluid samples of the patient, wherein (I) is earlier in IVIG therapy than (II),
wherein a greater or equivalent deviation from the control level of neopterin and the at least one biomarker selected from group (C) at time point (II) compared to the deviation from the control level of the biomarker at time point (I) indicates that the patient is non-responsive to IVIG therapy
and
   (ii) administering a therapeutical agent selected from the group consisting of glucocorticoids and biological agents to the patient.

A biological agent for use is provided in the treatment of a patient having MIS associated with an exposure to an infectious agent and insufficiently responding to IVIG therapy, wherein said use comprises the administration of the biological agent to the patient and wherein the patient is identified as insufficiently responding to IVIG therapy, comprising
(α) measuring the levels of neopterin at a time point (I) and a time point (II) in body fluid samples of the patient, and
(β) measuring levels of at least one biomarker selected from a group (C) consisting of: C1s-C1-INH complex, Bb, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP, complement classical pathway activity, complement alternative pathway activity, and properdin at a time point (I) and a time point (II) in body fluid samples of the patient,
wherein (I) is earlier in IVIG therapy than (II),
wherein a greater or equivalent deviation from the control level of C1s-C1-INH complex and the at least one biomarker selected from group (C) at time point (II) compared to the deviation from the control level of the biomarker at time point (I) indicates that the patient is non-responsive to IVIG therapy.

Preferably,
a higher or equivalent level of neopterin at time point (II) than at time point (I);
and a higher or equivalent level of the at least one biomarker selected from the group consisting of: Bb, C1s-C1-INH complex, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP,
and/or a lower or equivalent level of properdin at time point (II) than at time point (I);
and/or a greater or equivalent deviation from the control level of the at least one biomarker selected from the group consisting of complement classical pathway activity, complement alternative pathway activity at time point (II) compared to the deviation from the control level of the biomarker at time point (I), indicates that the patient is non-responsive to IVIG therapy.

In a preferred embodiment group (C) consists of: Bb, C1s-C1-INH complex, C3a, sC5b-9, C1-INH antigen, C1-INH activity, C4a, C4d, Factor B, Factor D, Factor H, Factor I, C-reactive protein, haptoglobin, troponin, ferritin, D-dimer, pro-BNP, Troponin T, pro-BNP and properdin.

In a preferred embodiment group (C) consists of: Bb, C1s-C1-INH complex, C3a, sC5b-9.

In any one of the second, third and fourth aspects

An altered level of the biomarker may be an elevated or increased level compared to a reference or control level. The reference or control level is preferably a level calculated from values of the biomarker characteristic of healthy individuals. The reference or control level is preferably the level of the biomarker characteristic of healthy individuals. The reference or control level or the values from which the reference or control level is calculated is/are preferably measured by the same method as the altered level. An altered level preferably means that the level is significantly different from the reference or control level, that is e.g. there is an at least 10% or at least 15% or at least 20% or at least 25% or at least 30% deviation from the reference or control level or preferably the altered level is statistically significantly different from the reference or control level.

In a highly preferred embodiment a C1s-C1-INH complex level measured in a subject that is at least 120% or at least 125% or at least 130% or at least 135% or most preferably at least about 140% of the reference level indicates that the subject has MIS.

In a highly preferred embodiment a Bb level measured in a subject that is at least 120%, at least 130%, at least 140%, at least 150%, at least 160% or most preferably at least about 175% or about 177% of the reference level indicates that the subject has MIS.

In a highly preferred embodiment a neopterin level measured in a subject that is at least 500%, at least 750%, at least 1000%, at least 1250% or most preferably at least about 1400% or 1450% of the reference level indicates that the subject has MIS.

In a highly preferred embodiment a measured C3a level that is at least 150% or at least 175% or at least 200% or most preferably at least 240% of the reference level indicates that the subject has MIS.

In a highly preferred embodiment a measured sC5b-9 level that is at least 150%, at least 175%, at least 200% or most preferably at least about 220% of the reference level indicates that the subject has MIS.

Preferably the measured level of the biomarker is compared to a reference or control level of the biomarker characteristic of healthy individuals, that is, individuals not having MIS or not at an increased risk of developing MIS or individuals who were not exposed to the infectious agent within 4 weeks, preferably within 6 weeks, preferably within 8 weeks or—where appropriate—to individuals who are already in remission from MIS. The reference or control level is preferably the reference or control level characteristic of the age of the subject to be tested.

The MIS might be MIS in children and adolescents (MIS-C) or in adults (MIS-A), preferably MIS-C.

The infectious agent is preferably a virus, more preferably Severe Acute Respiratory Syndrome-Coronavirus 2 (SARS-CoV-2).

Preferably the body fluid sample is blood sample, more preferably plasma sample, most preferably EDTA treated plasma sample.

In preferred embodiments the levels obtained in (α) and (β) are compared to a respective control level which is the same for time point (I) and time point (II),

Preferably, the biological agent is an anti-cytokine activity agent, preferably an interleukin [IL]-1 antagonist, an IL-6 receptor blocker or an anti-tumour necrosis factor agent. Preferably the anti-cytokine activity agent is selected from infliximab, anakinra and tocilizumab. Preferably the anti-cytokine activity agent is infliximab. Preferably the anti-cytokine activity agent is tocilizumab. Most preferably the anti-cytokine activity agent is anakinra.

Preferably the subject or patient was exposed to SARS-CoV-2 more than 1 week or more than 10 days or preferably more than 2 weeks, more preferably more than 3 weeks, preferably more than 4 weeks before the method is performed.

Preferably the subject or patient was exposed to SARS-CoV-2 less than 6 months, less than 5 months, less than 4 months and more preferably less than 3 months, preferably no more than 12, 11, 10, 9, 8, 7, 6, 5, 4, 3, 2, 1 weeks before the method is performed.

### In any One of the Aspects:

Preferably the subject or patient is 0-21 years of age, more preferably 0-19 years, more preferably 1-19 years, more preferably 1-16 years, most preferably 8-14 years.

Preferably the cut-off value of C1s-C1-inhibitor complex differentiating a patient having MIS-C or a subject at an increased risk of developing MIS-C from a healthy subject is about 1597 ng/mL.

Preferably the cut-off value of Bb differentiating a patient having MIS-C or a subject at an increased risk of developing MIS-C is about 1.39 μg/mL.

Preferably the cut-off value of C3a differentiating a patient having MIS-C or a subject at an increased risk of developing MIS-C is about 158 ng/mL.

Preferably the cut-off value of sC5b-9differentiating a patient having MIS-C or a subject at an increased risk of developing MIS-C is about 225 ng/mL Preferably the cut-off value of neopterin differentiating a patient having MIS-C or a subject at an increased risk of developing MIS-C is about 8.75 nmol/mL.

Preferably the cut-off value of C1s-C1-inhibitor complex differentiating a patient having active MIS-C from a a subject in remission after having MIS-C is about 1795 ng/mL.

Preferably the cut-off value of Bb differentiating a patient having active MIS-C from a a subject in remission after having MIS-C is about 1.11 μg/mL.

Preferably the cut-off value of C3a differentiating a patient having active MIS-C from a a subject in remission after having MIS-C is about 170 ng/mL.

Preferably the cut-off value of sC5b-9 differentiating a patient having active MIS-C from a a subject in remission after having MIS-C is about 363 ng/mL.

Preferably the cut-off value of neopterin differentiating a patient having active MIS-C from a a subject in remission after having MIS-C is about 10.7 nmol/mL.

The level of the biomarker can be measured by any means known in the art, e.g. by an immunoassay, e.g. by ELISA. The level of the biomarker can be determined by e.g. a commercially available or an in-house developed or prepared bioassay.

### Examples

In order to have a comprehensive view of the pathogenesis of MIS-C, we enrolled 34 acute, well-characterized MIS-C cases to this study, and took appropriate samples for detailed complement analysis before therapy. We measured multiple macrophage activation markers, inflammatory markers and multiple complement factor, regulator and activation product levels allowing us to draw conclusions on the presence and potential causes of complement activation and consumption. Twenty-eight of the 34 acute patients in the cohort were sampled also in remission making the paired analysis of complement profile changes along disease activity possible. Our results indicate the presence of complement activation in MIS-C with rapid normalization of activation product levels after IVIG treatment.

### Description of the MIS-C Cohort

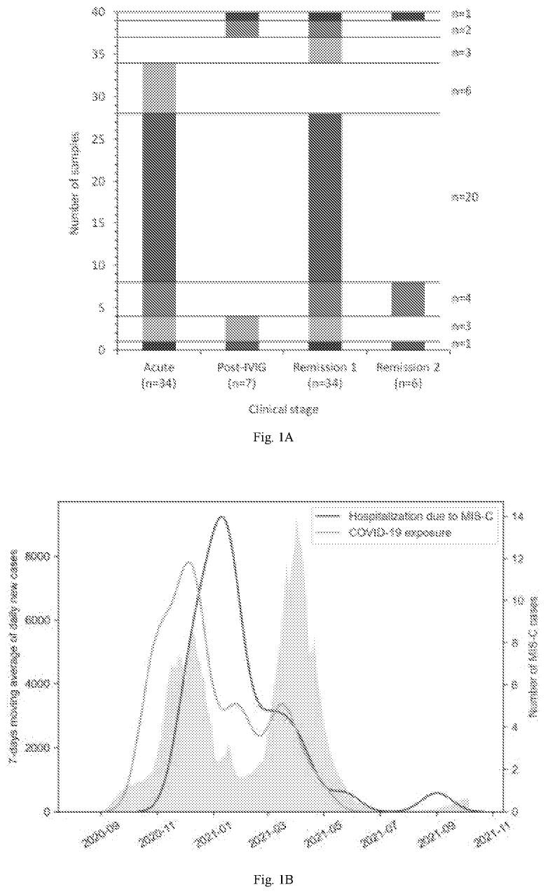

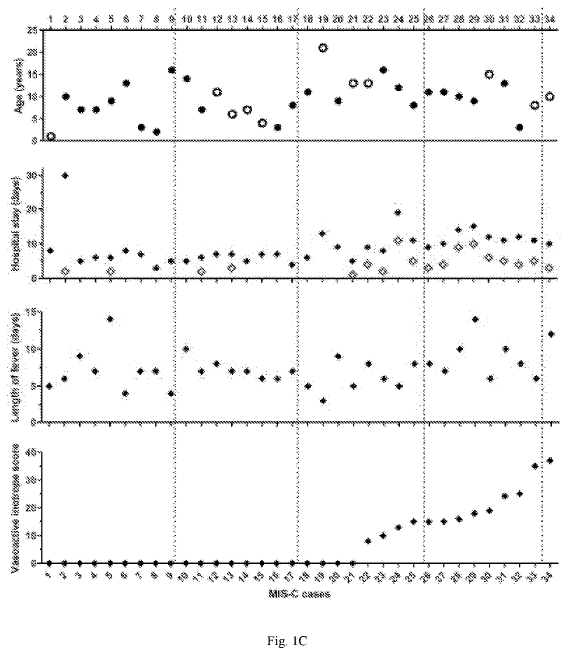

Between November 2020 and September 2021, we recruited 33 hospitalized children with acute MIS-C at Heim Pil National Pediatric Institute and one patient at the University of Szeged (FIG. 1A). All of the patients fulfilled the diagnostic criteria of MIS-C as defined by the World Health Organization (World Health Organization. Multisystem inflammatory syndrome in children and adolescents with COVID-19: Scientific Brief. 2020. Available at: https://www.who.int/publications-detail/multisystem-inflammatory-syndrome-in-children-and-adolescents-with-covid-19 (Accessed on May 17, 2020)). For 24/34 cases exposure (close contact) to COVID-19 patients was documented, MIS-C developed approximately 1 month later (median 32, 25th-75th percentile 28-44, range 8-89 days, FIG. 1B). Age distribution of the cohort was between 1 and 21 years, with peak around 10 years (median 9.5, 25th-75th percentile 7-13) with male predominance (23/34, 68%, FIG. 1C). Fifty-three percent (18/34, severe group) of the cases required pediatric intensive care unit (PICU) treatment (13/18 PICU cases also required vasopressor and/or inotrope treatment), whereas 47% (16/34) were mild-moderate cases (FIG. 1C). The pediatric healthy control group consisted of 18 children who underwent minor elective surgery (61% males, 8 (median), 6.5-13 (25th-75th percentile), 1-15 (range) years old).

All of the patients received 2 g/kg of body weight intravenous immunoglobulin, 97% of them low-dose aspirin (3-5 mg/kg, max 100 mg), 74% intravenous corticosteroids (2 mg/kg methylprednisolone) and 44% prophylactic low-molecular weight heparin treatment. None of the patients received second line immunomodulatory treatment. All 34 patients survived, 32 patients without long term sequelae at the time of last follow-up, although one patient with stroke and one with mesenterial thrombosis needed longer follow-up and rehabilitation.

Measurement of Neopterin levels was performed using a commercially available assay (IBL International GmbH).

Clinical laboratory tests were performed at clinical laboratories of institutions where the patients were treated, data were extracted from hospital records.

Measurement of anti-nucleocapsid and anti-spike SARS-CoV-2 IgM or IgG antibodies SARS-CoV-2 spike (S) and nucleocapsid (N) specific IgM or IgG were detected using commercially available assays (GA Generic Assays GmbH, Germany). The cut-off (Co) and binding index (BI) was calculated according to the manufacturer's instructions: [Cut-off (Co)=0.250+OD_{Negativecontol}, BI=OD sample/Co]. Samples with BI above 0.9 were considered positive.

Determination of anti-nucleocapsid or anti-spike SARS-CoV-2 IgG by research grade ELISA In-house ELISAs were performed with nucleocapsid (N) or full-length spike (S) or spike domain S1 or S2 recombinant proteins. Briefly, recombinant N or S were coated on 96-well polystyrene microtiter plates (Greiner Bio-One GmbH, Austria) at a concentration of 1 μg/ml in 100 μl coating bicarbonate buffer (pH9.8) at 4° C. overnight. After blocking with 1% bovine serum albumin (BSA) the plates were washed thoroughly with phosphate-buffered saline (PBS)-Tween. Patient and control sera were diluted 1:25 and loaded on the plates in duplicates. Plates were further incubated for 1 hour at room temperature and detected by a goat anti-human IgG secondary antibody labeled with HRP (SouthernBiotech). Absorbance values of samples and positive and negative controls were measured at 450 nm, with a reference wavelength of 620 nm using an automated plate reader (Tecan Group Ltd, Swit-zerland). Cut-off values were determined by the mean value plus 2 times the standard deviation (SD) of the negative control.

### Determination of IgG Subclasses Against the Full-Length Spike Protein

Briefly, recombinant S proteins and calibrators of purified human antibodies of each IgG subclass ranging from 1000 to 0 ng/ml were coated on 96-well polystyrene plates (Greiner Bio-One GmbH) overnight. After blocking, serum samples were added to the plate in dilutions between 1:25 and 1:250 and incubated for 1 hour at room temperature. The bound antibodies were then detected by HRP-conjugated mouse anti-human subclass specific antibodies. Cut-off values were determined by the mean OD value of 15.6 ng/ml IgG subclasses. Measurement of complement parameters

Commercially available ELISA kits were used to measure the concentrations of complement factors (factor D, properdin), biomarkers of complement activation (C4afragment, C4dfragment, C3afragment, Bb fragment, sC5b-9 complex), as well as lectin and alternative pathway activity and functional C1INH levels (C1INH activity). Measurements were conducted according to the manufacturers' instructions and the ELISA kits concerned are listed with the respective source and identifier number in the key resources table below.

Concentrations of C3 and C4 were measured by turbidimetry on a Beckman Coulter AU Chemistry Analyzer using the respective C3 and C4 reagents (Beckman Coulter, Brea, CA, USA).

Total classical pathway activity was measured by a hemolytic titration test based on Mayer's method (Fetterhoff and McCarthy: A micromodification of the CH50 test for the classical pathway of complement. J Clin Lab Immunol. 1984 August; 14(4):205-8.), using sheep erythrocytes sensitized by rabbit anti-sheep red blood cell antibodies. The antigenic concentrations of factor I, factor B and C1INH were measured by radial immunodiffusion using specific polyclonal antibodies (Reti et al. Complement activation in thrombotic thrombocytopenic purpura. J Thromb Haemost. 2012 May; 10(5):791-8.). Complement Clq and Factor H concentrations were determined by in-house sandwich ELISAs (Delamarche et al. An ELISA technique for the measurement of Clq in cerebrospinal fluid. J Immunol Methods. 1988 Nov. 10; 114 (1-2):101-6.; Reti et al supra). Antibodies used for the above measurements are listed in the key resources table (Table 2).

### Development of C1s-C1INH Complex and MASP1-C1INH Complex Assays

Novel immunoassay detecting levels of human C1s-C1INH complex and MASP1-C1INH in vitro were developed at Hycult Biotech in the Netherlands (Uden). The newly developed assays are not yet available commercially. The assays were performed according to manufacturer's instructions.

In brief, EDTA plasma samples and standards were incubated in wells coated with antibodies recognizing either activated C1s or MASP1. After incubation and washing, wells were incubated with an HRP-labeled antibody detecting bound C1INH in complex with the respective serine proteases. The amount of bound C1INH complexes was quantified by incubation with TMB substrate, before the enzymatic reaction was stopped by addition of oxalic acid after 15 minutes. Afterwards the absorbance was measured at 450 nm on a microplate reader and the concentration of C1INH complexes was determined based on a standard curve with known concentrations of either C1s-C1INH or MASP1-C1INH complex.

TABLE 2
Key resources table

| | | |
|---|---|---|
| REAGENT or RESOURCE | SOURCE | IDENTIFIER |

Antibodies

| | | |
|---|---|---|
| Mouse anti-human IgG1Fc-HRP (HP6001) | SouthernBiotech | 9054-05 |
| Mouse anti-human IgG2 Fc-HRP (31-7-4) | SouthernBiotech | 9060-05 |
| Mouse anti-human IgG3 Hinge-HRP (HP6050) | SouthernBiotech | 9210-05 |
| Mouse anti-human IgG4 Fc-HRP (HP6025) | SouthernBiotech | 9200-05 |
| Goat anti-human IgG HRP | SouthernBiotech | 2040-05 |
| Native human IgG1 protein | abcam | ab90283 |
| Native human IgG2 protein | abcam | ab90284 |
| Native human IgG3 protein | abcam | ab118426 |
| Anti-sheep red blood cell stroma antibody (rabbit) | Sigma-Aldrich | S1389 |
| Polyclonal antiserum to human factor B | Quidel | A311 |
| Polyclonal antiserum to human factor I | Quidel | A313 |
| Polyclonal antiserum to human C1 inhibitor (C1 esterase) | Quidel | A300 |
| Polyclonal antiserum to human C1q protein | Quidel | A301 |
| Monoclonal antibody to human factor H #1 | Quidel | A229 |
| Polyclonal rabbit anti-human C1q complement | Agilent Technologies | A013602 |
| Sheep anti-human factor H | Binding Site | PC030 |

Biological samples

| | | |
|---|---|---|
| Bovine serum albumin | Sigma-Aldrich | A3059 |

Chemicals, peptides, and recombinant proteins

| | | |
|---|---|---|
| Recombinant SARS-CoV-2 spike His protein, CF | R&D systems, biotecne | 10549-CV |
| Recombinant SARS-CoV-2 spike S1 subunit His-tag protein, CF | R&D systems, biotecne | 10569-CV-100 |
| Recombinant SARS-CoV-2 spike S2 subunit His-tag protein, CF | R&D systems, biotecne | 10594-CV-100 |
| Recombinant SARS-CoV-2 nucleocapsid His protein, CF | R&D systems, biotecne | 10474-CV-050 |

Critical commercial assays

| | | |
|---|---|---|
| MicroVue Bb Plus EIA Kit | Quidel | Cat# A027 |
| MicroVue sC5b-9 Plus EIA Kit | Quidel | Cat# A020 |
| MicroVue C4d Fragment EIA Kit | Quidel | Cat# A009 |
| MicroVue C4a Fragment EIA Kit | Quidel | Cat# A035 |
| MicroVue C3a Plus EIA Kit | Quidel | Cat# A032 |
| MicroVue C1-Inhibitor Plus EIA Kit | Quidel | Cat# A037 |
| Neopterin ELISA | IBL International GmbH | Cat# RE59321 |
| MASP1-C1INH complex, Human, ELISA Kit | Hycult Biotech | Cat# HK3001* |
| C1s-C1INH complex, Human, ELISA Kit | Hycult Biotech | Cat# HK399* |
| Complement factor D, Human, ELISA kit | Hycult Biotech | Cat# HK343 |
| Properdin, Human, ELISA kit | Hycult Biotech | Cat# HK334 |
| WIESLAB ® Complement System MBL Pathway | SVAR Life Science | Cat# COMPLMP320 |
| WIESLAB ® Complement System Alternative Pathway | SVAR Life Science | Cat# COMPLAP330 |
| GA CoV-2 IgG | GA Generic Assays | 3920 |
| GA CoV-2 IgM | GA Generic Assays | 3930 |
| Beckman Coulter C3 | Beckman Coulter | OSR6159 |
| Beckman Coulter C4 | Beckman Coulter | OSR6160 |

Software and algorithms

| | | |
|---|---|---|
| GraphPad Prism 9 software | GraphPadSoftwares Inc. | https://www.graphpad.com/ |
| | | scientific-soft- |
| | | ware/prism/ |
| Statistica 13.5 | TIBCO Softwares Inc. | https://www.tibco.com/ |

*not yet available commercially

TABLE 3
Diagnostic performance of selected biomarkers: Comparison of acute MIS-C
cases to healthy controls (verification of clinical diagnosis)

| | | | | |
|---|---|---|---|---|
| Marker | Cut-off point | Sensitivity | Specificity | Likelihood ratio* |

Comparison of acute MIS-C cases to healthy controls (verification of clinical diagnosis)

| | | | | | |
|---|---|---|---|---|---|
| Early classical | 1597 | ng/mL | 96.97 | 94.44 | 17.55 |
| pathway activation | | | 84.68-99.84 | 74.24-99.72 | (2.596-117.366) |
| marker C1s-C1- | | | | | |
| inhibitor complex | | | | | |
| Alternative path- | 1.39 | ug/mL | 90.9 | 100 | * |
| way activation | | | 76.43-96.86 | 82.41-100 | |
| product Bb | | | | | |
| Anaphylatoxin C3a | 158 | ng/mL | 92.86 | 94.12 | 15.786 |
| | | | 68.53-99.63 | 73.02-99.70 | (2.345-106.281) |
| Terminal pathway | 225 | ng/mL | 100 | 94.44 | 18.000 |
| activation product | | | 89.57-100 | 74.24-99.72 | (2.680-120.918) |
| sC5b-9 | | | | | |
| Macrophage | 8.75 | nmol/L | 100 | 100 | * |
| activation marker | | | 89.57-100 | 56.55-100 | |
| neopterin | | | | | |

TABLE 4
Diagnostic performance of selected biomarkers: Comparison of acute
MIS-C cases to remission (monitoring of therapy efficacy)

| | | | | |
|---|---|---|---|---|
| Marker | Cut-off point | Sensitivity | Specificity | Likelihood ratio* |

Comparison of acute MIS-C cases to remission (monitoring of therapy efficacy)

| | | | | | |
|---|---|---|---|---|---|
| Early classical | 1795 | ng/mL | 91.18 | 90.91 | 10.029 |
| pathway activation | | | 77.04-96.95 | 76.43-96.86 | (3.392-29.651) |
| marker C1s-C1-in- | | | | | |
| hibitor complex | | | | | |
| Alternative | 1.11 | ug/mL | 96.77 | 93.94 | 15.968 |
| pathway activation | | | 83.81-99.83 | 80.39-98.92 | (4.161-61.274) |
| product Bb | | | | | |
| Anaphylatoxin C3a | 170 | ng/mL | 100 | 85.71 | 7.000 |
| | | | 77.19-100 | 60.06-97.46 | (1.940-25.255) |
| Terminal pathway | 363 | ng/mL | 79.4 | 75.76 | 3.276 |
| activation product | | | 63.20-89.65 | 58.98-87.17 | (1.750-6.132) |
| sC5b-9 | | | | | |
| Macrophage | 10.7 | nmol/L | 100 | 100 | * |
| activation marker | | | 88.65-100 | 89.57-100 | |
| neopterin | | | | | |

*Likelihood ratio was calculated as sensitivity/(1-specificity), i.e. true positivity rate divided by the false positivity rate, indicating the diagnostic value of having a positive test result in supporting a certain condition (the diagnosis of MIS-C and the remission thereof, respectively).
For calculation of likelihood ratio positive result was considered in the following way: when comparing MIS-C cases to healthy controls, laboratory parameters above the cut-off were regarded as positive, whereas in the case of the acute-remission comparison, laboratory parameters below the cut-off were regarded as positive, as these values are supposed to indicate remission, which is the condition in question. Confidence intervals were calculated according to the method described by Simel at al. (Likelihood ratios with confidence: sample size estimation for diagnostic test studies. J Clin Epidemiol. 1991; 44(8): 763-70.).)
*Likelihood ratios cannot be calculated.

Macrophage Activation and Neutrophilia are Associated with Disease Severity in MIS-C

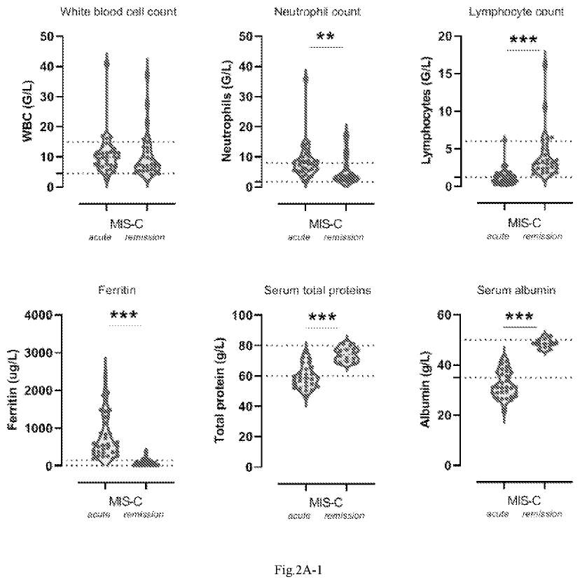

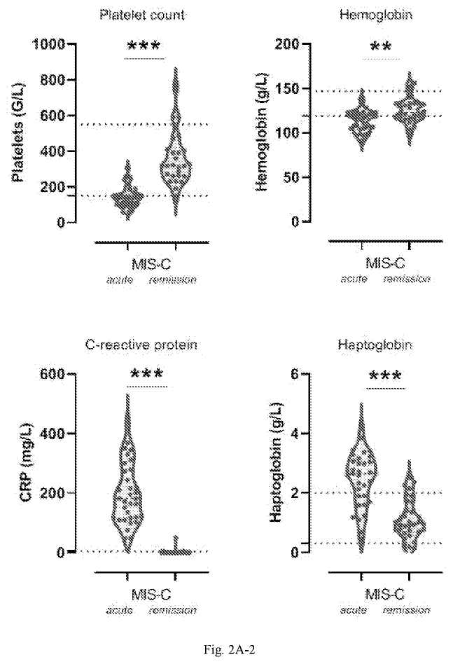

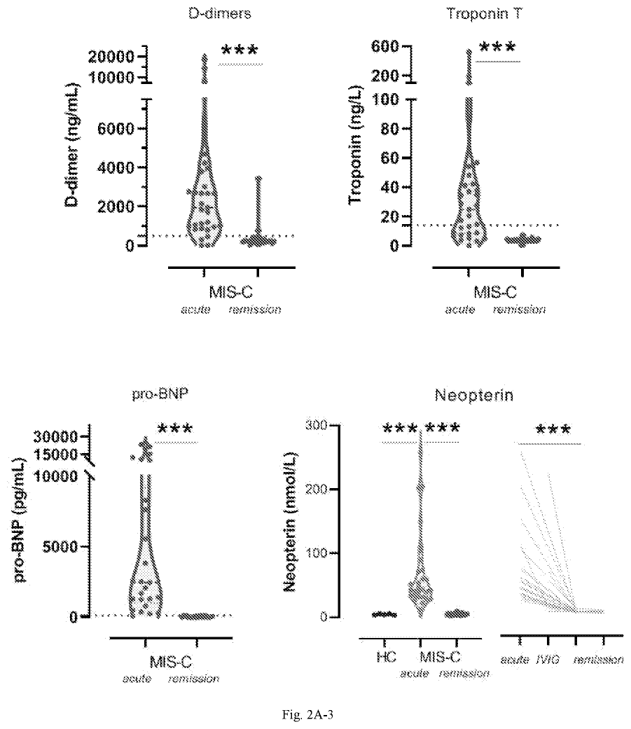

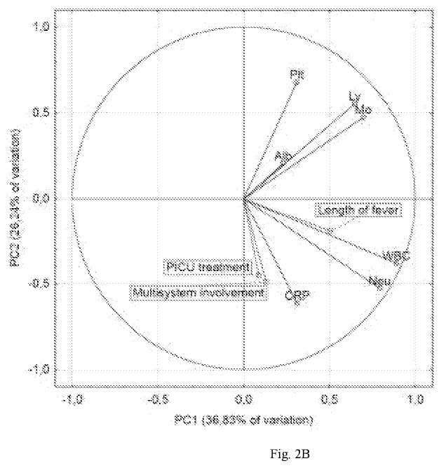

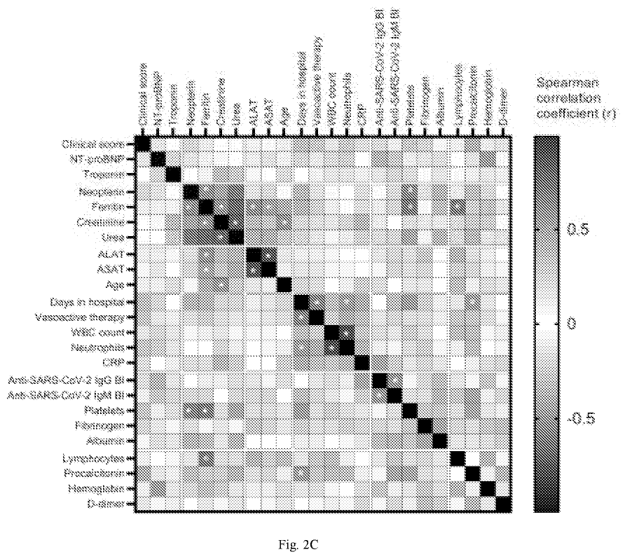

Absolute blood cell counts, disease activity, inflammatory and additional specific marker levels are presented on FIG. 2A. When compared to known reference ranges, the majority of the patients had elevated neutrophil- and decreased lymphocyte and platelet counts in acute stage. All acute cases showed marked elevations of C-reactive protein and ferritin levels, whereas for most of them haptoglobin, D-dimer, troponin and pro-BNP levels were also elevated, with lower total protein and albumin levels. This clearly supports the presence of severe, systemic inflammation and macrophage activation. The most striking difference between healthy children and acute MIS-C cases was noted for neopterin, a marker of macrophage activation. When investigated in remission, after IVIG therapy, all cell counts, inflammatory-, cardiac- and monocyte activation markers returned to the reference ranges supporting the presence of full disease remission (FIG. 2A).

With the help of principal component analysis, the overall architecture of severity markers was obtained (FIG. 2B). The first two components, explaining 63.07% of the variance in the data, showed that lymphocyte-, platelet-, monocyte- and albumin levels correlated with each other with CRP-, WBC- and neutrophil counts orthogonal to these variables. Interestingly, length of fever, requirement of PICU treatment, and extent of multisystemic involvement clustered together with CRP, WBC and neutrophil counts. FIG. 2C illustrates the relationships between clinical severity, cell counts and clinical laboratory markers. Neutrophilia shows association with number of days in hospital, whereas markers of monocyte activation (ferritin, neopterin) correlate with low platelet and lymphocyte counts and signs of kidney and hepatic involvement. Interestingly, whereas all patients were positive for IgG anti-SARS-CoV2 antibodies, their levels were not associated with clinical or laboratory markers of MIS-C severity.

High levels of complement activation markers are characteristic for acute MIS-C and show rapid decline after IVIG therapy

The complement profile was analyzed in a comprehensive manner in this study including measurement of all pathways' activities, components, regulators and specific activation products. This strategy allowed us to conclude on the presence, extent and potential causes of complement activation in MIS-C.

First, complement markers were compared between 18 healthy control children and 34 MIS-C cases in acute stage, before treatment. As presented on FIG. 3 (violin plots), acute MIS-C patients show significant elevation of complement components C4 and Factor B, of complement regulators C1-inhibitor, Factor H and Factor I. Activation products C1s-C1-inhibitor complex, C4a, C4d, C3a, Bb and sC5b-9 are significantly increased, whereas lectin pathway activation marker MASP1-C1-inhibitor complex is not. Interestingly, concentration of properdin, the positive regulator of alternative pathway (AP), was decreased, when compared to healthy controls. Functional activities of the lectin, classical and alternative pathways were similar in the two groups, without significant differences in levels of C1q, Factor D and C3. These results support the presence of hypercomplementemia with increased activation product levels of the classical, the alternative and terminal pathways.

Next, we analyzed changes of the complement biomarker levels in relation to disease stage. All patients received IVIG and went into clinical remission rapidly. Follow up samples were taken in remission for 34 patients (FIG. 1A), typically 1-2 months after hospital discharge. As shown in FIG. 3 (violin plots) levels of the complement components C4 and Factor B, and the complement regulators C1-inhibitor antigen and properdin concentrations normalized after treatment, as also observed for the activation products C1s-C1-inhibitor complex, C4a, C3a, Bb and sC5b-9. Split product C4d, Factor H and Factor I were the only markers without significant decrease in response to treatment. To give a global view on characteristic changes (direction, extent) of complement biomarkers, grey line charts in FIG. 3 show all measurement points for the MIS-C patients (for case/sample numbers see FIG. 1A), with p value markers indicating significance between the 28 acute- first remission sample pairs (paired analysis). This paired analysis shows again that levels of the complement components C4 and Factor B, the complement regulators C1-inhibitor antigen and properdin, and the activation products C1s-C1-inhibitor, C4a, C3a, Bb and sC5b-9 normalized after treatment, with most pronounced and uniform changes for the markers C1s-C1-inhibitor, Bb and C3a. Although activity of C1-inhibitor was not significantly elevated in acute MIS-C samples (when compared to healthy control, despite of the fact that antigenic expression was increased), C1-inh functional activity significantly declined in the MIS-C cohort in response to treatment. The comprehensive analysis of the complement biomarkers convincingly shows that remission of MIS-C is characterized by the normalization of the hyperactive complement system, parallel with the decline in additional inflammatory markers (FIG. 2A).

For 7 patients, samples taken 2-10 days after IVIG treatment were also available (group ‘IVIG’ on the line charts of FIG. 3). Remarkably, the most rapid, pronounced and uniform decline right after IVIG therapy, was noted for the markers C1s-C1-inhibitor, Bb, C3a and neopterin (FIGS. 3 and 2A, line charts). These results indicate that the normalization of complement and macrophage activation marker levels occur rapidly, within days after IVIG treatment.

Analysis of associations between complement profile, clinical severity, anti-SARS-CoV-2 antibodies, and inflammatory markers

In addition, the design of our study allowed investigation and identification of potential triggers and interactions underlying complement overactivation in acute MIS-C. We observed that levels of C4, C3, properdin, Factor I, Factor B and Factor H show positive correlations with functional activities of the classical and alternative pathways (FIG. 4A). Similarly strong positive associations were observed between activation markers of the alternative pathway (Bb), all pathways (C3a) and the terminal pathway (sC5b-9), indicating activation of the AP as the most closely related factor behind terminal pathway activation. Intriguingly, the early classical pathway marker C1s-C1-inhibitor complex level did not show any strong association with the functional activity of the classical pathway, or with the concentration of its specific component C1q. Split product C4a is the only factor whose level correlates with that of the C1s-C1-inhibitor complex (FIG. 4A).

Intriguingly, there was no significant correlation between any of the complement components, regulators or activation products and features of the anti-SARS-CoV2 humoral immune response including total IgG and IgM response (against spike (S)+nucleocapsid (N) protein antigens), or specific measures of anti-S, anti-N total IgG, or specific subclasses (IgG1 and IgG3). It is noteworthy that anti-SARS-CoV2 antibodies show no significant decline in response to IVIG treatment, except significant, non-uniform decrease of IgG binding index and anti-N IgG3 levels in remission (FIG. 5A). These observations make it unlikely that anti-SARS-CoV2 antibodies play a significant role as trigger factors behind complement activation in acute MIS-C.

Finally, clinical and laboratory markers of MIS-C severity and activity were investigated for their relationship with complement biomarkers in the acute disease stage. As shown on FIG. 4C, the global overview indicates rather low level, if any, of correlations between these factors. Only the markers Bb, C3a and sC5b-9 show a clear trend towards positive correlation with disease severity, including the significant positive correlation between Bb and ferritin and urea levels. In order to formally explore the potential contribution of key pathogenic features of MIS-C to the elevation of complement activation product levels, selected biomarkers of humoral anti-SARS-CoV-2 immune response, macrophage activation and hepatic acute phase reaction were further investigated (FIG. 5B). When patient groups are stratified according to the median levels of anti-SARS-CoV-2 IgG, ferritin or CRP levels, it is the alternative pathway activation marker Bb that shows a significant association with high levels of ferritin. The results collectively indicate that macrophage activation may have an important role in the activation of alternative pathway, and amplification of AP activation (C3a) with formation of the cell activating damaging sC5b-9 complex as part of the complex multisystem inflammatory response in MIS-C.

Performance of complement- and macrophage activation markers for diagnosis verification and therapy monitoring purposes in MIS-C

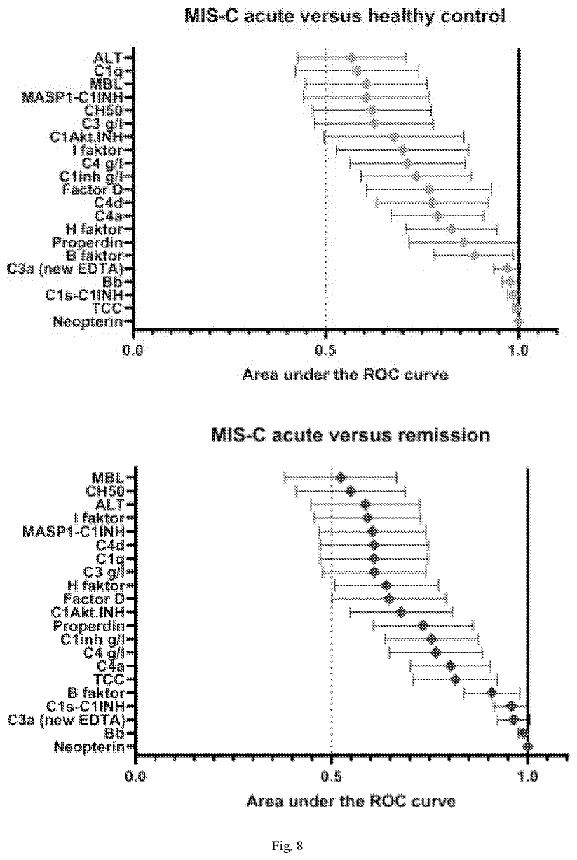

Lastly, we compared the capability of complement activation markers and neopterin to differentiate acute MIS-C cases from healthy children. Individual receiver operating characteristic (ROC) curves are presented on supporting FIG. 8, with calculated area under the ROC curves on FIG. 8. Complement activation markers C1s-C1-inhibitor, C3a, Bb, sC5b-9 and neopterin all had AUC value above 0.95, indicating paramount performance to support the diagnosis of MIS-C. Sensitivity and specificity values with likelihood ratios are summarized in Table 3. It is noteworthy that neopterin value above 8.75 nmol/L had 100% sensitivity and 100% specificity for the diagnosis of MIS-C, but all of the complement activation markers had similarly high sensitivity and specificity values, well above 90%. Cumulative frequencies of MIS-C cases and healthy controls are plotted against biomarker levels on FIG. 6A, clearly indicating the usefulness of these markers to support and verify the clinical diagnosis of MIS-C. The same markers were also evaluated for therapy monitoring purposes, comparing acute versus remission MIS-C cases. Individual receiver operating characteristic (ROC) curves are presented on FIG. 6B, where the best performing markers were neopterin, C1s-C1-inhibitor and Bb. All these markers had an area under the ROC curve (AUC) value near 1 (FIG. 6C), with sensitivity and specificity measures higher than 90% (Table 4.). Having for example neopterin value <10.7 nmol/L after therapy indicates remission with 100% sensitivity and specificity, with similarly prominent performance for C1s-C1-inhibitor and Bb (FIG. 6C and Table 4). These results make the use of these macrophage- and complement activation markers very promising for therapy monitoring purposes.

1-17. (canceled)

18. A method for treating a patient suffering in MIS associated with an exposure to an infectious agent who is insufficiently responding to IVIG therapy, comprising
(i) identifying the patient as insufficiently responding to IVIG therapy, comprising
(A) measuring the level of at least one biomarker that is C1s-C1-INH complex
at a time point (I) and a time point (II) in body fluid samples of the patient,
(B) comparing the levels obtained in (A) to a control level which is the same for time point (I) and time point (II),
wherein (I) is earlier in IVIG therapy than (II),
wherein a higher or equivalent level of the C1s-C1-INH complex at time point (II) than at time point (I) indicates that the patient is non-responsive to IVIG therapy and
(ii) administering a therapeutical agent selected from the group consisting of glucocorticoids and biological agents to the patient.

19-23. (canceled)

24. The method according to claim 18, wherein the at least one biomarker is neopterin, Bb and C1-INH complex.

25. The method according to claim 18, wherein the biological agent is an anti-cytokine agent.

26. The method according to claim 25, wherein the anti-cytokine agent is selected from the group consisting of interleukin [IL]-1 antagonists, IL-6 receptor blockers and anti-tumour necrosis factor agents.

27. The method according to claim 25, wherein the anti-cytokine agent is selected from the group consisting of infliximab, anakinra and tocilizumab.

28. The method according to claim 18, wherein the infectious agent is a virus.

29. The method according to claim 28, wherein the virus is Severe Acute Respiratory Syndrome-Coronavirus 2 (SARS-CoV-2).

30. The method according to claim 18, wherein the MIS is MIS-C.

31. The method according to claim 18, wherein the body fluid sample is plasma, preferably EDTA treated plasma.
